(19) United States
(12) Patent Application Publication
(10) Pub. No.: US 2020/0317603 A1
(43) Pub. Date: Oct. 8, 2020

(54) PROCESS FOR STABILIZATION OF AT LEAST MONOALKYL-SUBSTITUTED DIAMINOCYCLOHEXANES

(71) Applicant: BASF SE, Ludwigshafen am Rhein (DE)

(72) Inventors: Stephanie JAEGLI, Ludwigshafen (DE); Martin ERNST, Ludwigshafen (DE); Alexander PANCHENKO, Ludwigshafen (DE); Frank HETTCHE, Ludwigshafen (DE); Bjoern LUDOLPH, Ludwigshafen (DE)

(21) Appl. No.: 16/464,495

(22) PCT Filed: Nov. 27, 2017
(86) PCT No.: PCT/EP2017/080558

§ 371 Date: May 28, 2019

(30) Foreign Application Priority Data
Nov. 29, 2016 (EP) 16201152.2

(51) Int. Cl. C07C 209/90

(52) U.S. Cl. CPC C07C 209/90; C07C 211/36; C07C 2601/14

(57) ABSTRACT

A process for stabilizing monoalkyl-substituted diaminocyclohexanes, the process containing: adding a reductant and optionally water to a first composition containing a monoalkyl-substituted diaminocyclohexane and optionally water to obtain a second composition, wherein the second composition contains the reductant, the monoalkyl-substituted diaminocyclohexane and additionally at least 0.05% by weight of water based on the total weight of the second composition.

The present invention relates to a process for stabilization of at least monoalkyl-substituted diaminocyclohexanes in which at least one reductant (R) and optionally water are added to a composition (ZE). The composition (ZE) comprises at least one at least monoalkyl-substituted diaminocyclohexane (A) and optionally water. Addition of the at least one reductant (R) and optionally water to the composition (ZE) affords a composition (ZP) comprising at least 0.05% by weight of water based on the total weight of the composition (ZP). The invention also relates to the composition (ZP) and to the use thereof for example for producing hardeners for epoxy resins.

Compared to corresponding aromatic diamines, cycloaliphatic diamines such as alkyl-substituted diaminocyclohexanes generally have a better light-, UV- and weathering-stability and therefore represent important starting compounds for various products, for example coating materials. In pure form many cycloaliphatic amines are typically colorless liquids which, however, often have a propensity to discolor in the course of time on account of impurities originating for example from metals or metal compounds or from impurities of byproducts from the production process. The propensity for discoloration limits the field of application of cycloaliphatic amines and it is therefore desirable to effect appropriate purification of such materials and to prevent or minimize discoloration over the longest possible period. Since the formation of color-giving impurities is different in every production process for amines and in every amine product the decolorization and color-stabilization of some amines is not automatically also suitable for other amines.

The prior art therefore discloses in some cases very different approaches for decolorizing amines in the short term and improving the color stability thereof in the long term:

Thus for example U.S. Pat. No. 3,922,306 discloses a process for decolorizing aliphatic amines in which an aliphatic amine and an alkali metal borohydride are heated to 50 to 70° C. for several hours and subsequently separated from one another. According to U.S. Pat. No. 3,922,306 a corresponding decolorization could not be achieved by means of other reductants such as sodium sulfite, sodium dithionite, hydrazine or phosphinic acid.

U.S. Pat. No. 7,169,268 discloses a process for producing a color-stable tertiary amine in which a tertiary amine is distilled in the presence of ethylenediamine or an ethyleneamine derivative. The distilled tertiary amine discolors more slowly than corresponding untreated tertiary amine but the color stability investigations in U.S. Pat. No. 7,169,268 show that the discoloration of the amine increases after only a short time in air. A longer-term color stability may be achieved by storing the tertiary amine in an inert gas atmosphere.

U.S. Pat. No. 4,731,165 discloses a process for decolorizing triethylenetetramine according to which triethylenetetramine is catalytically purified with an ion exchanger resin based on sulfonic acid. The purification of the triethylenetetramine with the ion exchanger resin is effected under reduced pressure and elevated temperatures and following the purification step the ion exchanger resin is separated from triethylenetetramine by distillation. According to U.S. Pat. No. 4,731,165 the process is also suitable for purification of other polyalkylene polyamines but does not contain any information concerning the longer-term color stability of the amines.

U.S. Pat. No. 5,362,914 describes a continuous process for reducing the discoloration of polyethylene polyamines in which polyethylene polyamines are hydrogenated at elevated temperatures and elevated pressure in the presence of a cobalt-, copper- and chromium-containing catalyst in a hydrogen atmosphere. The polyethylene polyamines may be distilled before the hydrogenation or employed as crude product and the hydrogenation is followed by a further distillation step. Although the process described in U.S. Pat. No. 5,362,914 is suitable for decolorizing polyethylene polyamines, the thus obtained polyethylene polyamines also have a propensity for discoloration in air and according to U.S. Pat. No. 5,362,914 should be stored under a nitrogen atmosphere.

An alternative process for decolorizing polyethylene polyamines is disclosed in U.S. Pat. No. 4,609,436 in which polyethylene polyamines are admixed with a chlorine-containing hydrocarbon and stirred at elevated temperatures. The treatment of the polyethylene polyamines with the chlorine-containing hydrocarbon is followed by a distillation in which the polyethylene polyamine is separated from the chlorine-containing hydrocarbon.

U.S. Pat. No. 6,774,264 discloses a process for improving the color stability of N,N-dialkylalkanolamines by hydrogenation of corresponding amines in the presence of a palladium catalyst under aqueous conditions. In U.S. Pat. No. 6,774,264 the hydrogenation is effected predominantly for removal of unsaturated byproducts formed during production of N,N-dialkylalkanolamines.

U.S. Pat. No. 5,847,221 discloses a process for decolorizing alkanolamines or alkyleneamines in which the corresponding amine is treated with a polymeric solid acid catalyst in the presence of small amounts of water at elevated temperatures and elevated pressure for several hours to remove or to decompose metal catalyst residues from the respective production process of the amines and compounds having conjugated double bonds. The decolorizing step may be followed by a distillation step.

U.S. Pat. No. 5,359,139 discloses a process for treatment of tertiary amines in which a tertiary amine is admixed with ascorbic acid which is subsequently separated from the tertiary amine again by distillation. In the thus treated tertiary amines a discoloration of the tertiary amines under acidic conditions may thus be very largely avoided.

While the previously recited processes describe options for instant purification of various amines compositions comprising amines having long-term color stability are generally obtainable through the use of certain stabilizers.

Thus for example U.S. Pat. No. 4,602,108 discloses the color stabilization of linear or branched aliphatic amines by suitable stabilizers such as nitrilotrismethylenephosphonic acid, 8-hydroxyquinoline or ethylenediaminetetraacetic acid as a stabilizer. The stabilizer has only low to poor solubility in the aliphatic amine.

WO 2011/084865 discloses a composition comprising an oxidation-sensitive amine and an oxidation inhibitor. The oxidation inhibitor may be a free radical scavenger such as for example phenylimidazole or glutamine or an antioxidant such as ascorbic acid. The use of the oxidation inhibitor is especially intended to prevent the formation of formaldehyde and dimethylformamide which are often formed in the oxidation-sensitive amines described in WO 2011/084865 during storage in air.

“Color-Stabilized DYTEK® DCH-99 amine Adducts for Epoxy Curing, Invista, Mar. 14, 2012” discloses the stabilization of 1,2-diaminocyclohexane which comprises a stabilizer system composed of sodium borohydride, water, benzyl alcohol, triethylamine and an epoxy resin to avoid discoloration of the amine during storage in air. Additional amounts of sodium borohydride and water increase the color stability. The weight fraction of the stabilizer system is markedly greater than the weight fraction of the 1,2-diaminocyclohexane.

It is an object of the present invention to provide a process for stabilization of at least monoalkyl-substituted diaminocyclohexanes.

This object is achieved by a process for stabilization of at least monoalkyl-substituted diaminocyclohexanes comprising addition of at least one reductant (R) and optionally water to a composition (ZE) comprising at least one at least monoalkyl-substituted diaminocyclohexane (A) and optionally water to obtain a composition (ZP), wherein the composition (ZP) comprises at least 0.05% by weight of water based on the total weight of the composition (ZP).

It was found that, surprisingly, the discoloration of at least monoalkyl-substituted diaminocyclohexanes during storage in air may be markedly reduced when before storage thereof at least one reductant (R) and optionally water are added.

Both thermal stress and relatively lengthy storage of the stabilized at least monoalkyl-substituted diaminocyclohexanes in air result in markedly less severe color deepening than for unstabilized at least monoalkyl-substituted diaminocyclohexanes.

Without wishing to be bound to a particular theory the at least one reductant (R) can prevent the formation of condensation products of the at least monoalkyl-substituted diaminocyclohexanes or of any other amines present in the composition (ZE) and also of color-giving descendent products thereof which can form over time through storage of at least monoalkyl-substituted diaminocyclohexanes in air.

In addition the process according to the invention does not require the removal of water which may be present in the at least monoalkyl-substituted diaminocyclohexanes as a residue from the corresponding production processes, thus rendering complex and costly separation processes for removal of water from the at least monoalkyl-substituted diaminocyclohexanes unnecessary.

In the context of the present invention the terms “(color) stability”, “(color) stabilization” or “(color) stable” are to be understood as meaning that the color number of an amine or an amine-containing composition remains unchanged at a low level over a relatively lengthy period or increases comparatively slightly. Determination of color number is effected via long-term storage tests in which the color quality of a compound is determined by measurement of the transmission of incident light. To this end a solution of a particular concentration is irradiated with a light beam of defined wavelength in a cuvette having a known wall thickness. The percentage of transmitted light energy gives a defined color number at a given wavelength. Commonly used for weakly colored solutions is the determination of the Hazen color number according to the APHA platinum-cobalt color scale. Determination of the Hazen color number according to the APHA platinum-cobalt color scale is generally performed according to DIN EN ISO 6271.

In the context of the present invention the term “condensation product” comprises all compounds formed in a reaction with elimination of water, ammonia, carbon dioxide, hydrogen halides or alcohols.

The present invention is elucidated in detail hereinbelow.

The present invention relates to a process for stabilization of at least monoalkyl-substituted diaminocyclohexanes.

In the process according to the invention at least one reductant (R) is added to a composition (ZE) comprising at least one at least monoalkyl-substituted diaminocyclohexane as component (A).

The composition (ZE) is the mixture comprising the at least one at least monoalkyl-substituted diaminocyclohexane (A). The at least one at least monoalkyl-substituted diaminocyclohexane (A) may be present in the composition (ZE) in any desired amount. All of the following statements concerning the composition (ZE) therefore relate to the corresponding mixture before the addition of the at least one reductant (R).

The composition (ZE) comprises by preference at least 69% by weight, preferably at least 90% by weight and particularly preferably at least 94% by weight of the at least one at least monoalkyl-substituted diaminocyclohexane (A) based on the total weight of the composition (ZE).

In a further embodiment the composition (ZE) comprises by preference at least 85% by weight, preferably at least 90% by weight and particularly preferably at least 95% by weight of the at least one at least monoalkyl-substituted diaminocyclohexane (A) based on the total weight of the composition (ZE).

In the context of the present invention the at least one at least monoalkyl-substituted diaminocyclohexane (A) is to be understood as meaning a compound comprising a cyclohexane ring having two amino groups and having at least one or more alkyl substituents.

Alkyl-substituted diaminocyclohexanes are known in principle to those skilled in the art and may be produced by any processes known to those skilled in the art. Suitable processes for producing alkyl-substituted diaminocyclohexanes comprise for example the metal-catalyzed hydrogenation of alkyl-substituted diaminobenzene derivatives, such as is disclosed for example in WO 2009/090179 and WO 2009/153123.

The at least one at least monoalkyl-substituted diaminocyclohexane (A) is preferably at least one at least monoalkyl-substituted 1,2-, 1,3- or 1,4-diaminocyclohexane, in particular at least one at least monoalkyl-substituted 1,3-diaminocyclohexane.

The at least one at least monoalkyl-substituted diaminocyclohexane (A) is preferably selected from one of the compounds according to general formulae (I), (II) or (III)

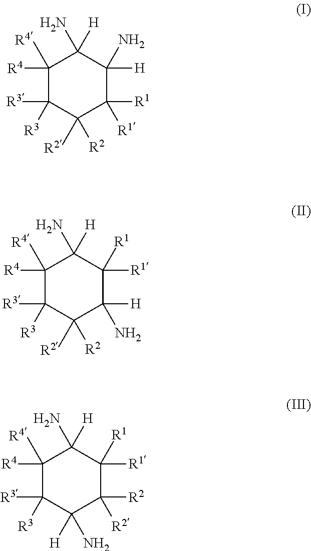

in which
R₁, R₁′, R₂, R₂′, R₃, R₃′, R₄ and R₄′ are independently of one another selected from H and C₁-C₄-alkyl, wherein at least one radical R₁, R₁′, R₂, R₂′, R₃, R₃′, R₄ or R₄′ is C₁-C₄-alkyl.

In the context of the present invention the designation C₁-C₄-alkyl, such as is used for example for the radical R¹ in general formulae (I), (II) and (III), is to be understood as meaning that this substituent is an alkyl radical having 1 to 4 carbon atoms. The alkyl radical may be either linear or branched. Examples of alkyl radicals are methyl, ethyl, n-propyl, n-butyl and branched isomers thereof.

It is preferable when R₁, R₁′, R₂, R₂′, R₃, R₃′, R₄ and R₄′ are independently of one another selected from H or methyl.

The at least one at least monoalkyl-substituted diaminocyclohexane (A) preferably conforms to general formula (I), wherein R₁, R₁′, R₂, R₂′, R₃, R₃′, R₄ and R₄′ are independently of one another selected from H or C₁-C₄-alkyl, wherein precisely one radical R₁, R₁′, R₂, R₂′, R₃, R₃′, R₄ or R₄′ is C₁-C₄-alkyl.

It is particularly preferable when the at least one at least monoalkyl-substituted diaminocyclohexane (A) is selected from 1,3-diamino-4-methylcyclohexane or 1,3-diamino-2-methylcyclohexane.

The at least one at least monoalkyl-substituted diaminocyclohexane (A) may be precisely one at least monoalkyl-substituted diaminocyclohexane or else mixtures of two or more different at least monoalkyl-substituted diaminocyclohexanes.

In a preferred embodiment the at least one at least monoalkyl-substituted diaminocyclohexane (A) is a mixture of 1,3-diamino-4-methylcyclohexane and 1,3-diamino-2-methylcyclohexane. The weight fractions of 1,3-diamino-4-methylcyclohexane and 1,3-diamino-2-methylcyclohexane in this mixture may in principle take any desired values. It is preferable when the at least one at least monoalkyl-substituted diaminocyclohexane (A) in this preferred embodiment comprises 50% to 95% by weight of 1,3-diamino-4-methylcyclohexane and 5% to 50% by weight of 1,3-diamino-2-methylcyclohexane based on the total weight of the at least one at least monoalkyl-substituted diaminocyclohexane (A).

In a further preferred embodiment the at least one at least monoalkyl-substituted diaminocyclohexane (A) is selected from 1,3-diamino-2-methylcyclohexane or 1,3-diamino-4-methylcyclohexane.

Furthermore, the composition (ZE) may optionally comprise water. In principle the water may have been introduced into the composition (ZE) in any desired manner. The water may for example be a residue from the production process of the at least one at least monoalkyl-substituted diaminocyclohexane (A).

It is preferable when the composition (ZE) has a water content of at least 0.05% by weight, preferably of at least 0.08% by weight and particularly preferably of at least 0.1% by weight based on the total weight of the composition (ZE).

It is further preferable when the composition (ZE) has a water content of not more than 1% by weight, preferably of not more than 0.8% by weight and particularly preferably of not more than 0.5% by weight based on the total weight of the composition (ZE).

In a preferred embodiment the composition (ZE) has a water content of 0.05% to 1% by weight, preferably of 0.08% to 0.8% by weight and particularly preferably of 0.1% to 0.5% by weight based on the total weight of the composition (ZE).

The composition (ZE) may comprise not only the at least one at least monoalkyl-substituted diaminocyclohexane (A) and optionally water but also further compounds.

The further compounds are preferably residues from the respective production processes of the corresponding at least one at least monoalkyl-substituted diaminocyclohexane (A). These residues comprise for example unsubstituted or at least monoalkyl-substituted aminocyclohexanes, impurities due to metals and metal compounds of hydrogenation catalysts, higher boiling byproducts or solvent residues such as for example of isopropanol, isobutanol, tert-butanol, dimethoxyethane, dioxane or tetrahydrofuran.

Higher boiling byproducts are to be understood as meaning those constituents having a higher boiling point than the at least one at least monoalkyl-substituted diaminocyclohexane (A), wherein the boiling point of the higher boiling byproducts is preferably at least 2° C., particularly preferably at least 4° C. and very particularly preferably at least 6° C. higher than the standard boiling point of the at least one at least monoalkyl-substituted diaminocyclohexane (A). In the context of the present invention the term “standard boiling point” is to be understood as meaning the boiling point at standard pressure of 1.013 bar.

When two or more at least monoalkyl-substituted diaminocyclohexanes (A) are present in the composition (ZE) the boiling point of each higher boiling byproduct is higher than the highest boiling point of the two or more at least monoalkyl-substituted diaminocyclohexanes (A).

The higher boiling byproducts preferably have a molecular weight in the range from 100 to 500 g/mol, more preferably 120 to 370 g/mol and particularly preferably 150 to 300 g/mol.

The higher boiling byproducts comprise preferably at least one cyclohexane fragment (radical), more preferably two cyclohexane fragments (radicals) or at least one cyclohexane fragment (radical) and at least one aromatic fragment (radical).

These comprise for example unsubstituted or at least monoalkyl-substituted aromatic amines and secondary amines which may be formed as condensation products of two or more molecules of the at least one at least monoalkyl-substituted diaminocyclohexane (A) by elimination of ammonia, for example N¹-(3-amino-4-methylcyclohexyl)-4-methylcyclohexane-1,3-diamine and isomers thereof. The higher boiling byproducts furthermore also comprise condensation products of the at least one at least monoalkyl-substituted diaminocyclohexane (A) with other compounds present in the composition (ZE) and comprising amino groups, for example with unsubstituted or at least monoalkyl-substituted aromatic amines or optionally unsubstituted or at least monoalkyl-substituted monoaminocyclohexanes.

Furthermore, storage of the at least one at least monoalkyl-substituted diaminocyclohexane (A) in air may over time through oxidation reactions result in formation of higher boiling byproducts which may be involved in the discoloration of the at least one at least monoalkyl-substituted diaminocyclohexane (A) in the composition (ZE). These comprise for example imino compounds and olefinically unsaturated compounds which may be formed by oxidation of the abovementioned condensation products.

Such compounds comprise for example oxidized condensation products such as N-(3-imino-4-methylcyclohexyl)-4-methylcyclohex-1-ene-1-amine and isomers thereof and oxidized condensation products of the at least one at least monoalkyl-substituted diaminocyclohexane (A) with other compounds present in the composition (ZE) and comprising amino groups.

It is preferable when the higher boiling byproducts are selected from unsubstituted or at least monoalkyl-substituted aromatic amines or secondary amines, imines and/or olefinically unsaturated compounds formed by condensation reactions and optionally oxidation reactions of the at least one at least monoalkyl-substituted diaminocyclohexane (A) optionally with further amines present in the composition (ZE).

It is particularly preferable when the higher boiling byproducts are selected from N¹-(3-amino-4-methylcyclohexyl)-4-methylcyclohexane-1,3-diamine or N-(3-imino-4-methylcyclohexyl)-4-methylcyclohex-1-ene-1-amine and isomers thereof.

In one embodiment the composition (ZE) comprises
95% to 99.999% by weight of the at least one at least monoalkyl-substituted diaminocyclohexane (A),
0.001% to 5% by weight of higher boiling compounds,
based on the total weight of the composition (ZE), wherein the sum of all components in the composition (ZE) makes 100% by weight.

In a further embodiment the composition (ZE) comprises
94% to 99.949% by weight of the at least one at least monoalkyl-substituted diaminocyclohexane (A),
0.05% to 1% by weight of water,
0.001% to 5% by weight of higher boiling compounds,
based on the total weight of the composition (ZE), wherein the sum of all components in the composition (ZE) makes 100% by weight.

It is preferable when the further compounds present in the composition (ZE) are removed before the addition of the at least one reductant (R) is effected (see below).

In the process according to the invention the composition (ZE) is admixed with at least one reductant (R) to obtain the composition (ZP).

This addition of the at least one reductant (R) may be effected by all methods known to those skilled in the art and is preferably effected with stirring.

The addition of the at least one reductant (R) may in principle be effected at any desired temperature. It is preferable when the addition of the at least one reductant (R) is performed at low temperatures, preferably in the range from 5° C. to 60° C. and particularly preferably in the range from 10° C. to 40° C.

The duration of the addition of the at least one reductant (R) may in principle be effected within a very wide timespan. The duration of the addition of the at least one reductant (R) is preferably in the range from 10 minutes to 8 hours, particularly preferably in the range from 15 minutes to 5 hours and particularly preferably in the range from 20 minutes to 3 hours. Those skilled in the art will select the duration of the addition of the at least one reductant (R) appropriately to obtain a homogenous solution of the composition (ZP).

The composition (ZP) is the mixture comprising the at least one reductant (R), the at least one at least monoalkyl-substituted diaminocyclohexane (A) and additionally at least 0.05% by weight of water based on the total weight of the composition (ZP). Accordingly, all statements concerning the composition (ZP) relate to the mixture after the addition of the at least one reductant (R) and optionally water.

The at least one reductant (R) may comprise precisely one reductant or else mixtures of two or more different reductants. The at least one reductant (R) preferably comprises at least one component comprising hydride ions.

It is preferable when the at least one reductant (R) comprises at least one hydride ion-containing boron or aluminum compound. Such compounds are known in principle to those skilled in the art.

The at least one reductant (R) is particularly preferably selected from lithium borohydride, sodium borohydride, potassium borohydride, sodium cyanoborohydride, lithium aluminum hydride, sodium aluminum hydride or potassium aluminum hydride. The at least one reductant (R) is very particularly preferably sodium borohydride.

In the composition (ZP) the at least one reductant (R) may in principle be present in any amounts commonly used and known to those skilled in the art for stabilizers. The composition (ZP) preferably comprises 0.005% to 0.2% by weight of the at least one reductant (R) based on the total weight of the composition (ZP). The composition (ZP) comprises preferably 0.007% to 0.15% by weight and particularly preferably 0.01% to 0.1% by weight of the at least one reductant (R) based on the total weight of the composition (ZP).

The at least one reductant (R) may be added in the form of a solid, in a solution (L) or in a suspension (S). If the at least one reductant (R) is added in a solution (L) or in a suspension (S) the solution (L) or the suspension (S) may in principle comprise any desired solvent. It is preferable when the solution (L) or the suspension (S) comprise water, amines, ethers or alcohols as solvent and it is particularly preferable when the solution (L) or the suspension (S) comprise water as solvent.

The at least one reductant (R) is preferably present in the composition (ZP) in fully dissolved form. This means that the composition (ZP) preferably comprises no solid particles of the at least one reductant (R). Accordingly, the at least one reductant (R) preferably cannot be removed from the composition (ZP) by filtration.

Similarly, reaction products of the at least one reductant (R) with any higher boiling byproducts present in the composition (ZP) or with oxidants such as oxygen, preferably atmospheric oxygen, are preferably present in the composition (ZP) in fully dissolved form. This means that the composition (ZP) preferably comprises no solid particles of the abovementioned reaction products of the at least one reductant (R) either. Accordingly, the abovementioned reaction products of the at least one reductant (R) preferably cannot be removed from the composition (ZP) by filtration either.

In one embodiment the at least one reductant (R) is added in a solution (L), wherein the solution (L) preferably comprises at least one basic compound (B). In a further embodiment the at least one reductant (R) is added in a suspension (S), wherein the suspension (S) preferably comprises at least one basic compound (B). All of the following preferences for the solution (L) apply correspondingly to the suspension (S).

The solution (L) may comprise precisely one basic compound (B) or else mixtures of two or more different basic compounds (B).

The amount of the at least one basic compound (B) in the solution (L) is not decisive for the process according to the invention. The solution (L) preferably comprises 0.1% to 75% by weight of the at least one basic compound (B) based on the total weight of the solution (L). The solution (L) preferably comprises 5% to 70% by weight and particularly preferably 10% to 65% by weight of the at least one basic compound (B) based on the total weight of the solution (L).

The at least one basic compound (B) may in principle be any basic compound known to those skilled in the art. It is preferable when the at least one basic compound (B) is a basic alkali metal or alkaline earth metal compound. It is particularly preferable when the at least one basic compound (B) is selected from lithium hydroxide, sodium hydroxide, potassium hydroxide, magnesium hydroxide, calcium hydroxide, barium hydroxide, lithium carbonate, sodium carbonate, potassium carbonate, magnesium carbonate or calcium carbonate. It is very particularly preferable when the at least one basic compound (B) is selected from sodium hydroxide, potassium hydroxide or sodium carbonate.

In a preferred embodiment the mixture (G) comprises the following components:

a) 5% to 20% by weight of the at least one reductant (R),
b) 10% to 65% by weight of the at least one basic compound (B) and
c) 15% to 85% by weight of water,
wherein the weight fractions of the components a), b) and c) altogether sum to 100% by weight.

As mentioned previously, especially storage of the at least one at least monoalkyl-substituted diaminocyclohexane (A) in air may over time result in formation of higher boiling byproducts which are involved in the discoloration of the at least one at least monoalkyl-substituted diaminocyclohexane (A).

A preferred embodiment therefore comprises initially performing a distillation of the composition (ZE) to remove higher boiling byproducts and to obtain a distilled composition (DZE) comprising the at least one at least monoalkyl-substituted diaminocyclohexane (A) and optionally water followed by the addition of the at least one reductant (R) and optionally water to the distilled composition (DZE) to obtain the composition (ZP), wherein the composition (ZP) comprises at least 0.05% by weight of water based on the total weight of the composition (ZP).

For the sake of completeness it is noted that in this embodiment the at least one reductant (R) is added to the distilled composition (DZE) and not to the composition (ZE).

The distillation may be effected according to any method which is known to those skilled in the art and based on the respective embodiment judged to be technically appropriate.

The distillation may be effected for example inter alia in a rotary evaporator, a distillation column, by Kugelrohr distillation or short-path distillation.

The distillation may also be effected in two or more steps by one distillation technique or by a combination of different distillation techniques and may be effected continuously or discontinuously.

The distillation may in principle be performed in air or in the absence of oxygen. To avoid undesired oxidation reactions through the at least one at least monoalkyl-substituted diaminocyclohexane (A) or other compounds present in the composition (ZE) during the distillation the distillation is preferably effected in the absence of oxygen. “In the absence of oxygen” is to be understood in the context of the present invention as meaning that the volume fraction of oxygen in the distillation apparatus is less than 0.1% by volume, preferably less than 0.1% by volume and particularly preferably less than 0.01% by volume based on the total volume of the distillation apparatus.

The distillation may in principle be effected at any desired temperature. The distillation is preferably effected at a temperature in the range from 70° C. to 180° C., preferably in the range from 80° C. to 170° C. and particularly preferably in the range from 90° C. to 160° C.

The distillation may in principle be effected at any desired pressure. The distillation is preferably effected at a pressure in the range from 0.1 to 500 mbar, preferably in the range from 0.5 to 300 mbar and particularly preferably in the range from 1 to 100 mbar.

In a preferred embodiment the distillation is effected at a temperature in the range from 70° C. to 180° C., preferably in the range from 80° C. to 170° C. and particularly preferably in the range from 90° C. to 160° C. and at a pressure in the range from 0.1 to 500 mbar, preferably in the range from 0.5 to 300 mbar and particularly preferably in the range from 1 to 100 mbar.

The composition (ZP) obtained via the process according to the invention comprises at least 0.05% by weight of water based on the total weight of the composition (ZP).

In a preferred embodiment the composition (ZP) comprises by preference 0.05% to 3% by weight, preferably 0.10% to 2% by weight and particularly preferably 0.15% to 1.5% by weight of water based on the total weight of the composition (ZP).

In principle the water may have been introduced into the composition (ZP) in any desired manner.

In one embodiment at least a portion of the water present in the composition (ZP) is already present in the composition (ZE), wherein the composition (ZE) preferably comprises a water content of 0.05% to 1% by weight, preferably of 0.08% to 0.8% by weight and particularly preferably of 0.1% to 0.5% by weight based on the total weight of the composition (ZE). As mentioned previously, the water may for example be a residue from the production process of the at least one at least monoalkyl-substituted diaminocyclohexane (A).

If a distillation is effected before the addition of the at least one reductant (R) then the water present in the composition (ZE) is preferably not removed completely. In the case where the composition (ZE) has a water content of 0.05% to 1% by weight, preferably of 0.08% to 0.8% by weight and particularly preferably of 0.1% to 0.5% by weight based on the total weight of the composition (ZE) it is particularly preferable when the distillation does not effect any removal of water whatsoever.

In the case where the composition (ZE) has a water content of more than 1% by weight based on the total weight of the composition (ZE) the distillation preferably removes a quantity of water such that the distilled composition (DZE) comprises not more than 1% by weight, preferably not more than 0.8% by weight and particularly preferably 0.5% by weight of water based on the total weight of the composition (DZE).

In a further embodiment the process according to the invention additionally comprises the addition of water. This addition of water may be effected by all methods known to those skilled in the art and is preferably effected with stirring.

The addition of water may be effected together with the at least one reductant (R) or else before or after the addition of the at least one reductant (R).

The water that is added may in principle be any desired water, for example demineralized water or singly or multiply distilled water.

If the at least one reductant (R) is added in a solution (L) comprising water as solvent or in a suspension (S) comprising water as solvent the further addition of water may in principle be eschewed. However, it is preferable when the addition of water is effected even when the at least one reductant (R) was added in a solution (L) or in a suspension (S).

In a further embodiment at least a portion of the water present in the composition (ZP) is already present in the composition (ZE) and the process according to the invention additionally comprises the addition of water.

The weight ratio of water to the at least one reductant (R) in the composition (ZP) is by preference at least 1:1, preferably at least 2:1 and particularly preferably at least 4:1.

The weight ratio of water to the at least one reductant (R) in the composition (ZP) is furthermore by preference not more than 100:1, preferably not more than 50:1 and particularly preferably not more than 30:1.

In a preferred embodiment the weight ratio of water to the at least one reductant (R) in the composition (ZP) is by preference 100:1 to 1:1, preferably 50:1 to 2:1 and particularly preferably 30:1 to 4:1.

The water is preferably present in the composition (ZP) in fully mixed form. This is to be understood as meaning that the composition (ZP) preferably comprises no separate water phases. Accordingly, the water present in the composition (ZP) preferably cannot be removed from the composition (ZP) by phase separation.

The present invention further provides the composition (ZP) produced by the process according to the invention.

The composition (ZP) preferably comprises the following components:
96.8% to 99.945% by weight of at least one at least monoalkyl-substituted diaminocyclohexane (A),
0.005% to 0.2% by weight of at least one reductant (R) and
0.05% to 3% by weight of water.

The at least monoalkyl-substituted diaminocyclohexanes stabilized by the process according to the invention may be used as synthesis building blocks for the production of surfactants, pharmaceutical and plant protection products, stabilizers, light stabilizers, polymers, isocyanates, hardeners for epoxy resins, catalysts for polyurethanes, intermediates for producing quaternary ammonium compounds, plasticizers, corrosion inhibitors, synthetic resins, ion exchangers, textile auxiliaries, dyes, vulcanization accelerators, emulsifiers and/or as starting materials for the production of ureas and polyureas.

The present invention accordingly also provides for the use of the composition (ZP) for producing surfactants, pharmaceutical and plant protection products, stabilizers, light stabilizers, polymers, isocyanates, hardeners for epoxy resins, catalysts for polyurethanes, intermediates for producing quaternary ammonium compounds, plasticizers, corrosion inhibitors, synthetic resins, ion exchangers, textile auxiliaries, dyes, vulcanization accelerators, emulsifiers and/or as starting materials for the production of ureas and polyureas.

In particular, 1,3-diamino-2-methylcyclohexane and 1,3-diamino-4-methylcyclohexane may be used as monomer building blocks for polyamides, as hardeners for epoxy resins or as starting products for the production of the corresponding isocyanates.

The following examples are intended to more particularly elucidate the present invention but without limiting the present invention thereto.

Determination of the APHA Hazen color number in the examples which follow is effected according to DIN EN ISO 6271 and is performed in a Lange (LICO 400) colorimeter. Determination of Hazen color number is effected in Lange LZM 130 50 mm single use plastic cuvettes. The measured samples each have a mass of 60 g.

### EXAMPLE 1

Samples of an undistilled composition (ZE) comprising a mixture of 1,3-diamino-2-methylcyclohexane and 1,3-diamino-4-methylcyclohexane and 0.15% by weight of water are filled into 250 mL screwtop glass jars, admixed with sodium borohydride and optionally water to obtain a composition (ZP) and stored in a drying cabinet at 80° C. (see experiments 1 and 2 in table 1).

Also, 895 g of the composition (ZE) are distilled via a distillation bridge at 1 mbar and 104° C. (top temperature). 805 g of a distilled composition (DZE) comprising approximately 0.15% by weight of water are obtained. Samples of this distilled composition (DZE) are likewise filled into 250 mL screwtop glass jars, admixed with sodium borohydride and optionally water to obtain a composition (ZP) and stored in a drying cabinet at 80° C. (see experiments 3 to 9 in table 1).

For reference an undistilled sample (V1) not admixed with sodium borohydride and a distilled sample (V2) not admixed with sodium borohydride are stored and analyzed. All samples are regularly opened and aerated and per measurement about 6 g are withdrawn for the color number determination.

The corresponding experimental data are reported in table 1. Reported in the columns is the Hazen color number at the respective times recited in the uppermost row.

TABLE 1

| | NaBH₄ | Water | | | | | | | | |
|---|---|---|---|---|---|---|---|---|---|---|
| | [% by | addition | | | | | | | | |
| Experiment | wt.] | [% by wt.] | 0 h | 24 h | 48 h | 72 h | 96 h | 168 h | 192 h | 240 h |
| V1 | — | — | 12 | 29 | 48 | 78 | 149 | 318 | 683 | >1000 |
| V2 | — | — | 0 | 0 | 5 | 15 | 31 | 89 | 204 | 509 |
| 1 | 0.05 | — | 12 | 48 | 77 | 103 | 106 | 220 | 282 | 385 |
| 2 | 0.05 | +0.3 | 12 | 29 | 54 | 79 | 49 | 66 | 135 | 261 |
| 3 | 0.1 | +0.5 | 0 | 58 | 92 | 122 | 128 | 96 | 72 | 67 |
| 4 | 0.1 | +1.0 | 0 | 15 | 38 | 45 | 54 | 22 | 23 | 74 |
| 5 | 0.1 | +1.5 | 0 | 10 | 17 | 12 | 44 | 25 | 68 | 178 |
| 6 | 0.05 | +0.2 | 0 | 51 | 92 | 100 | 92 | 127 | 140 | 188 |
| 7 | 0.05 | +0.5 | 0 | 56 | 69 | 75 | 85 | 56 | 59 | 167 |
| 8 | 0.05 | +1.0 | 0 | 12 | 28 | 58 | 49 | 23 | 34 | 226 |
| 9 | 0.01 | — | 0 | 15 | 24 | 32 | 32 | 22 | 29 | 121 |

The experimental data quoted in table 1 show that the Hazen color number of the composition (ZP) can be kept markedly lower in the long term when the at least one reductant (R) and optionally water are employed (experiments 1 to 9). When the composition (ZE) was initially distilled and subsequently admixed with the at least one reductant (R) and optionally water (see experiments 3 to 9) the Hazen color number is lower over a longer period than for undistilled stabilized compositions (ZP) (see experiments 1 and 2).

### EXAMPLE 2

Samples of an undistilled composition (ZE) comprising a mixture of 1,3-diamino-2-methylcyclohexane and 1,3-diamino-4-methylcyclohexane and 0.15% by weight of water are filled into 250 mL screwtop glass jars, admixed with sodium borohydride/Borol™ (12.5% by weight sodium borohydride in an aqueous, 14 molar solution of sodium hydroxide obtainable from Dow Chemicals) and optionally water to obtain a composition (ZP) and stored in a drying cabinet at 80° C. (see experiments 1 and 2 in table 2).

Also, 860 g of the composition (ZE) were distilled via a distillation bridge at 1 mbar and 104° C. (top temperature). 730 g of a distilled composition (DZE) comprising approximately 0.15% by weight of water are obtained. Samples of this distilled composition (DZE) are likewise filled into 250 mL screwtop glass jars, admixed with sodium borohydride/Borol and optionally water to obtain a composition (ZP) and stored in a drying cabinet at 80° C. (see experiments 3 to 7 in table 2).

For reference a distilled sample (experiment V3) not admixed with a reductant (R) is stored and analyzed. Since the undistilled composition (ZE) is identical to the undistilled composition (ZE) from example 1 the reference measurement of the undistilled sample not admixed with a reductant (R) corresponds to the experiment V1 from example 1. All samples are regularly opened and aerated and per measurement about 6 g are withdrawn for the color number determination.

In the experiments 1 to 5 Borol™ is employed as the reductant (R) and in the experiments 6 and 7 sodium borohydride is employed as the reductant (R). The reported weight fractions of the at least one reductant (R) in table 2 which follows always relate to the employed amount of sodium borohydride. When for example Borol™ is used as the reductant (R) then the designation “0.02% by weight” is to be understood as meaning that a certain amount of Borol™ is employed until 0.02% by weight of sodium borohydride have been added by means of Borol™.

The corresponding experimental data are reported in table 2. Reported in the columns is the Hazen color number at the respective times recited in the uppermost row.

TABLE 2

| | | Water | | | | | | | | |
|---|---|---|---|---|---|---|---|---|---|---|
| | Reductant | addition | | | | | | | | |
| | (R) | [% by | | | | | | | | |
| Experiment | [% by wt.] | wt.] | 0 h | 24 h | 48 h | 72 h | 96 h | 168 h | 192 h | 240 h |
| V1 | — | — | 12 | 29 | 48 | 78 | 149 | 318 | 683 | >1000 |
| V3 | — | — | 0 | 1 | 3 | 12 | 30 | 102 | 251 | 538 |
| 1 | 0.02 | — | 0 | 71 | 116 | 155 | 208 | 368 | 487 | 653 |
| 2 | 0.04 | — | 0 | 105 | 97 | 97 | 130 | 412 | 360 | 501 |
| 3 | 0.01 | — | 0 | 8 | 14 | 15 | 12 | 24 | 33 | 152 |
| 4 | 0.02 | — | 0 | 10 | 17 | 22 | 16 | 20 | 35 | 218 |
| 5 | 0.04 | — | 0 | 32 | 63 | 72 | 83 | 67 | 83 | 185 |
| 6 | 0.02 | +0.1 | 0 | 19 | 38 | 39 | 52 | 34 | 41 | 115 |
| 7 | 0.04 | +0.2 | 0 | 39 | 89 | 94 | 100 | 75 | 70 | 177 |

The experimental data quoted in table 2 show that the Hazen color number of the composition (ZP) can be kept markedly lower in the long term when the at least one reductant (R) and optionally water are employed (experiments 1 to 7). When the composition (ZE) is initially distilled and subsequently admixed with the at least one reductant (R) and optionally water (see experiments 3 to 7) the Hazen color number is lower over a longer period than for undistilled stabilized compositions (ZP) (see experiments 1 and 2).

### EXAMPLE 3

449 g of an undistilled composition (ZE) comprising 99.76% by weight of a mixture of 1,3-diamino-2-methylcyclohexane and 1,3-diamino-4-methylcyclohexane and 0.24% by weight of further compounds are distilled at 70 mbar and 136° C. (top temperature). 51 g of a first fraction are obtained and separated and subsequently 343 g of a distilled composition (DZE) are obtained. This distilled composition (DZE) comprises no water.

Samples of this distilled composition (DZE) are filled into 250 mL screwtop glass jars, admixed with a reductant (R) and stored in a drying cabinet at 80° C.

For reference a distilled sample (experiment V1) not admixed with a reductant (R) is stored and analyzed. All samples are regularly opened and aerated and per measurement about 6 g are withdrawn for the color number determination.

In experiment V2 Borol™ (12.5% by weight sodium borohydride in an aqueous, 14 molar solution of sodium hydroxide obtainable from Dow Chemicals) is employed as the reductant (R) and in experiment V3 sodium borohydride is employed as the reductant (R). The reported weight fractions of the at least one reductant (R) in table 3 which follows always relate to the employed amount of sodium borohydride. When Borol™ is used as the reductant (R) then the designation “0.1% by weight” for example is to be understood as meaning that a certain amount of Borol™ is employed until 0.1% by weight of sodium borohydride have been added by means of Borol™. When using Borol™ the water content of the samples is always below 0.1% by weight.

The corresponding experimental data are reported in table 3. Reported in the columns is the Hazen color number at the respective times recited in the uppermost row.

TABLE 3

| Ex- | Reductant | | | | | | | | |
|---|---|---|---|---|---|---|---|---|---|
| peri- | (R) | | | | | | | | |
| ment | [% by wt.] | 0 h | 24 h | 48 h | 72 h | 96 h | 168 h | 192 h | 240 h |
| V1 | — | 0 | 0 | 3 | 11 | 32 | 78 | 159 | 305 |
| V2 | 0.1 | 0 | 3 | 9 | 45 | 104 | 253 | 402 | 791 |
| V3 | 0.1 | 1 | 96 | 123 | 195 | 260 | 339 | 837 | >1000 |

The experimental data quoted in table 3 show that for a low Hazen color number in the long term not only the at least one reductant (R) but also at least 0.05% by weight of water based on the total weight of the composition (ZP) must be present in the composition (ZP).

### EXAMPLE 4

Samples of an undistilled composition (ZE) comprising a mixture of 1,3-diamino-2-methylcyclohexane and 1,3-diamino-4-methylcyclohexane and 0.15% by weight of water are filled into 250 mL screwtop glass jars, admixed with sodium borohydride or a sodium borohydride stock solution to obtain a composition (ZP) and stored in a drying cabinet at 60° C. (see experiments 1 and 2 in table 4).

For reference an undistilled sample (V1) not admixed with sodium borohydride is stored and analyzed. All samples are regularly opened and aerated and per measurement about 6 g are withdrawn for the color number determination.

The sodium borohydride stock solution is produced as follows: In a 100 mL stirred flask fitted with a bubble counter and under nitrogen 2 g of NaBH₄ powder are initially charged and subsequently 18 g of the composition (ZE) are added thereto. After one hour of stirring the NaBH₄ is except for a few crystals almost fully dissolved. The supernatant solution is used as NaBH₄ stock solution.

The corresponding experimental data are reported in table 4. Reported in the columns is the Hazen color number at the respective times recited in the uppermost row.

TABLE 4

| | | Addition | | | | | | | | | | | | |
|---|---|---|---|---|---|---|---|---|---|---|---|---|---|---|
| | NaBH₄ | Stock | | | | | | | | | | | | |
| | [% by | solution | | | | | | | | | | | | |
| Experiment | wt.] | [% by wt.] | 0 h | 24 h | 48 h | 72 h | 144 h | 168 h | 192 h | 312 h | 336 h | 360 h | 384 h | 408 h |
| V1 | — | — | 21 | 27 | 36 | 47 | 89 | 106 | 128 | 233 | 277 | 326 | 386 | 454 |
| 1 | 0.01 | — | 21 | 41 | 36 | 35 | 48 | 57 | 67 | 107 | 122 | 141 | 171 | 183 |
| 2 | — | 0.01 | 21 | 38 | 32 | 32 | 51 | 60 | 70 | 111 | 129 | 147 | 172 | 198 |

The experimental data quoted in table 4 show that the Hazen color number of the composition (ZP) can be kept markedly lower in the long term when sodium borohydride or a sodium borohydride stock solution are employed (experiments 1 and 2).

1. A process for stabilizing at least monoalkyl-substituted diaminocyclohexanes, the process comprising:
adding at least one reductant and optionally water to a first composition comprising at least one at least monoalkyl-substituted diaminocyclohexane and optionally water to obtain a second composition,
wherein the second composition comprises the at least one reductant, the at least one at least monoalkyl-substituted diaminocyclohexane and additionally at least 0.05% by weight of water based on a total weight of the second composition.

2. The process of claim 1, wherein the at least one at least monoalkyl-substituted diaminocyclohexane is a compound of formula (I), (II) or (III) 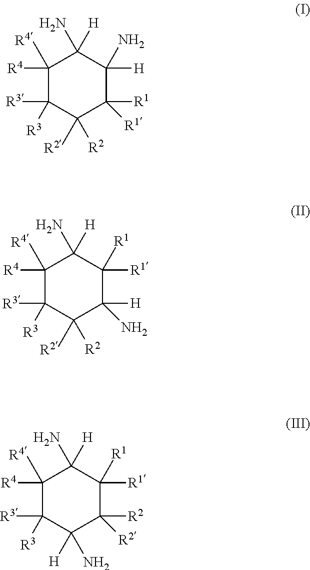
wherein R₁, R₁′, R₂, R₂′, R₃, R₃′, R₄ and R₄′ are independently of one another selected from the group consisting of H and C₁-C₄-alkyl, and
at least one of radical R₁, R₁′, R₂, R₂′, R₃, R₃′, R₄ and R₄′ is C₁-C₄-alkyl.

3. The process of claim 1, wherein the at least one at least monoalkyl-substituted diaminocyclohexane is selected from the group consisting of 1,3-diamino-4-methylcyclohexane and 1,3-diamino-2-methylcyclohexane.

4. The process of claim 1, wherein the first composition comprises at least 85% by weight of the at least one at least monoalkyl-substituted diaminocyclohexane based on a total weight of the first composition.

5. The process of claim 1, wherein the at least one reductant is selected from the group consisting of lithium borohydride, sodium borohydride, potassium borohydride, sodium cyanoborohydride, lithium aluminum hydride, sodium aluminum hydride and potassium aluminum hydride.

6. The process of claim 1, wherein the second composition comprises 0.005 to 0.2% by weight of the at least one reductant based on the total weight of the second composition.

7. The process of claim 1, wherein the adding comprises adding the at least one reductant in the form of a solid, in a solution or in a suspension.

8. The process of claim 7, wherein a mixture comprises at least one basic compound selected from the group consisting of lithium hydroxide, sodium hydroxide, potassium hydroxide, ammonium hydroxide, magnesium hydroxide, calcium hydroxide, barium hydroxide, lithium carbonate, sodium carbonate, potassium carbonate, magnesium carbonate and calcium carbonate.

9. The process of claim 7, wherein the solution comprises:
a) 5% to 20% by weight of the at least one reductant,
b) 10% to 65% by weight of at least one basic compound and
c) 15% to 85% by weight of water,
wherein weight fractions of components a), b) and c) altogether sum to 100% by weight.

10. The process of claim 1, further comprising:
initially performing a distillation of the first composition to remove higher boiling byproducts and to obtain a distilled composition comprising the at least one at least monoalkyl-substituted diaminocyclohexane and optionally water, and
adding the at least one reductant and optionally water to the distilled composition to obtain the second composition,
wherein the second composition comprises at least 0.05% by weight of water based on the total weight of the second composition.

11. The process of claim 10, wherein
i) the distillation is effected at a temperature in a range from 70° C. to 180° C., and/or
ii) the distillation is effected at a pressure in a range from 0.1 to 500 mbar.

12. The process of claim 1, wherein
i) at least a portion of the water present in the second composition is already present in the first composition, wherein the first composition comprises a water content of 0.05% to 1% by weight based on a total weight of the first composition, and/or
ii) the process further comprises adding water, and/or
iii) at least a portion of the water present in the second composition is added together with the at least one reductant.

13. The process of claim 1, wherein the second composition comprises 0.05% to 3% by weight of water based on the total weight of the second composition.

14. The process of claim 1, wherein a weight ratio of water to the at least one reductant in the second composition is 100:1 to 1:1.

15. A second composition, comprising the second composition comprises the at least one reductant, the at least one at least monoalkyl-substituted diaminocyclohexane and additionally at least 0.05% by weight of water based on a total weight of the second composition.

16. An article, for comprising:
the second composition of claim 15,
wherein the article is selected from the group consisting of a surfactant, a pharmaceutical and plant protection product, a stabilizer, a light stabilizer, a polymer, an isocyanate, a hardener for epoxy resin, a catalyst for polyurethane, an intermediate for producing a quaternary ammonium compound, a plasticizer, a corrosion inhibitor, a synthetic resin, an ion exchanger, a textile auxiliary, a dye, a vulcanization accelerator and an emulsifier.
